(19) United States
(12) Patent Application Publication
(10) Pub. No.: US 2008/0234286 A1
(43) Pub. Date: Sep. 25, 2008

(54) STABLE AMORPHOUS IMATINIB MESYLATE AND PRODUCTION PROCESS THEREFOR

(75) Inventors: Alex Weisman, Kiriat Ekron (IL); Sonia Krivonos, Beer Sheva (IL); Edna Danon, Meitar (IL); Itai Adin, Beer Sheva (IL); Carmen Iustain, Beer Sheva (IL)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: CHEMAGIS Ltd., Bnei Brak (IL)

(21) Appl. No.: 12/051,597

(22) Filed: Mar. 19, 2008

Related U.S. Application Data
(60) Provisional application No. 60/895,840, filed on Mar. 20, 2007.

(51) Int. Cl. A61K 31/506; C07D 401/14

(52) U.S. Cl. 514/252.18; 544/295

(57) ABSTRACT

Provided herein is a spray dried stable amorphous imatinib mesylate as a free-flowing solid and process for producing the amorphous imatinib mesylate in highly pure form.

### BACKGROUND OF THE INVENTION

Imatinib (N-{5-[4-(4-methyl-piperazinomethyl)-benzoylamido]-2-methylphenyl}-4-(3-pyridyl)-2-pyrimidine-amine) is represented by the following structural formula (I):

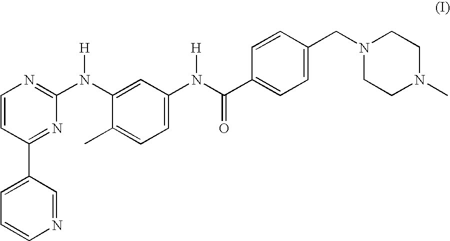

Imatinib is known as an inhibitor of tyrosine kinases and is indicated for the treatment of chronic myeloid leukemia (CML), Philadelphia chromosome positive leukemia, for patients in chronic phase and in blast crisis, accelerated phase and also for malignant gastrointestinal stromal tumors. It selectively inhibits activation of target proteins involved in cellular proliferation. Imatinib also has potential for the treatment of other cancers that express these kinases, including acute lymphocytic leukemia and certain solid tumors. Imatinib is sold by Novartis as Gleevec™ or Glivec® capsules containing imatinib mesylate equivalent to 100 mg of imatinib free base.

U.S. Pat. No. 6,894,051 (to Novartis AG), hereinafter the '051 patent, describes two crystalline forms of imatinib mesylate, the α-form and the β-form. It is mentioned in the '051 patent that the α-form is hygroscopic and that it is characterized by needle-shaped crystals, which make them “not particularly well-suited to pharmaceutical formulation as solid dosage forms, because their physical properties, for example their flow characteristics, are unfavorable”. According to the '051 patent, the a-form may be obtained by precipitating out the imatinib mesylate salt from a solution in an organic solvent such as methanol. Thus, it is concluded in the '051 patent that the β-form is preferred over other crystalline forms and/or the amorphous form of imatinib mesylate.

Patent application WO 2007/023182 (to Novartis AG), hereinafter the '182 application, describes two additional crystalline forms of imatinib mesylate, that is the δ-form and the ε-form. It is mentioned in the '182 application that the 6-form and the ε-forms “have advantageous utilities and properties”.

Application WO 2006/054314 describes two crystalline forms of imatinib mesylate, designated as forms I and II, and processes for obtaining these two forms.

Since each polymorph may have different characteristic behavior, a major problem of using a crystalline polymorphic drug is associated with obtaining a reproducible solid form of the active pharmaceutical ingredient. An example of the limitations associated with polymorphs is the anti-epilepsy drug carbamazepine, in which only a specific crystalline form is allowed, because the US Pharmacopoeia dictates in the monograph the pharmaceutical use of only a specific crystalline form (characterized by its X-ray diffraction pattern). In addition, other health authorities require assurance for the correct crystalline form of the drug used as well.

One way of alleviating the problem, of obtaining reproducible solid forms of active pharmaceutical ingredients, may be using non-crystalline forms of these materials. On one hand the problem of having variety of crystalline forms does not exist. On the other hand, amorphous solids are known to have better dissolution. As a result, one can expect a good, consistent availability of the active ingredient. Therefore, non-crystalline materials may be offered as a solution to this problem because when a material is amorphous, there cannot be polymorphism. Typically, such a non-crystalline form has a better solubility and faster dissolution rate, thus assuring good bioavailability.

U.S. Pat. No. 7,300,938 (hereinafter the '938 Patent) describes a crystalline form of imatinib mesylate, designated as form Hi, and processes for obtaining this form. According to the teachings of the '938 Patent, the imatinib mesylate designated as form H1, is prepared from chlorinated solvents such as chloroform and dichloromethane by methods which are less preferable for industrial implementation, because of the hazards concerned in using these solvents.

Other forms provided by the '938 Patent are crystalline imatinib mesylate hydrate and amorphous imatinib mesylate hydrate. According to Examples 6 and 8 of the '938 Patent, the amorphous imatinib mesylate hydrate is prepared by dissolving imatinib mesylate form H1 in a 5:1 mixture of methanol and water and subjecting the solution to vacuum drying and spray drying respectively. A skilled artisan can expect that by carrying out vacuum drying of a 5:1 solution of imatinib mesylate in methanol:water, a sticky and very hydroscopic solid will be obtained. Using methanol on large-scale production can be problematic because it is a volatile and poisonous solvent, so that spray drying from aqueous solution is much preferable because there is no solvent hazard involved in this process whatsoever.

Furthermore, in an article by G. E. Taylor et al. published in Journal of Chromatography A. 1119 (2006) 231-237, it is mentioned that “Sulfonic acids such as methanesulfonic acid (mesylate) are often used during manufacture of pharmaceuticals, either as counter-ions to form a salt, as acid catalyst or as the result of protecting group removal during the synthesis. However, the presence of any alcohol either in the stages of synthesis or in the crystallization stage of the salt may cause the formation of sulfonic acid esters which are considered to be alkylating agents. In fact, methyl and ethyl methanesulfonate esters are known genotoxins and are known carcinogens in rats and mice”.

Thus, the methods of obtaining amorphous imatinib mesylate, as detailed in the '938 Patent, are not practical for pharmaceutical use because of the possible formation of the methyl ester of methanesulfonic acid.

The amorphous imatinib mesylate described in the '938 Patent is allegedly a hydrate form, containing between 2-3.2% water, while the calculated water content of a hydrate is 2.96%. However, a hydrate is defined as a solid compound containing water molecules combined in a definite ratio as an integral part of the crystal. Therefore, amorphous material, which lacks the crystalline structure, cannot form hydrates.

Furthermore, an amorphous imatinib mesylate hydrate, obtained by evaporation drying, might tend to be lumpy and therefore its industrial usage in formulating the API into the pharmaceutical composition can be limited. Therefore, there is a need in the art for an amorphous stable imatinib mesylate, which has a suitable particle distribution for usage in the pharmaceutical compositions containing it.

### SUMMARY OF THE INVENTION

In one embodiment, the present invention provides a process for preparing stable amorphous imatinib mesylate, which includes the steps of:

dissolving imatinib mesylate in water to obtain a solution;

optionally filtering the solution; and

removing the water.

According to an embodiment of the present invention, the said removing can be by a method selected from evaporation, drying, e.g., in an oven, spray drying or freeze drying, preferably spray drying or freeze drying.

The water content in the spray dried amorphous imatinib mesylate of the present invention is in the range of 3.2-5% water.

According to the present invention, highly pure spray dried amorphous imatinib mesylate is obtained, having a purity of at least 98.5%, preferably having a purity equal to or greater than 99.5%.

Thus, the best mode of practicing the invention is by spray drying an aqueous solution of imatinib mesylate, using specific spray drying parameters, to obtain stable amorphous imatinib mesylate having narrow particle distribution, which is suitable for pharmaceutical compositions containing the amorphous imatinib mesylate.

### BRIEF DESCRIPTION OF THE FIGURES

FIG. 1 depicts the X-ray powder diffraction pattern of amorphous imatinib mesylate (example 4).

FIG. 2 depicts the infrared spectrum of amorphous imatinib mesylate (example 4).

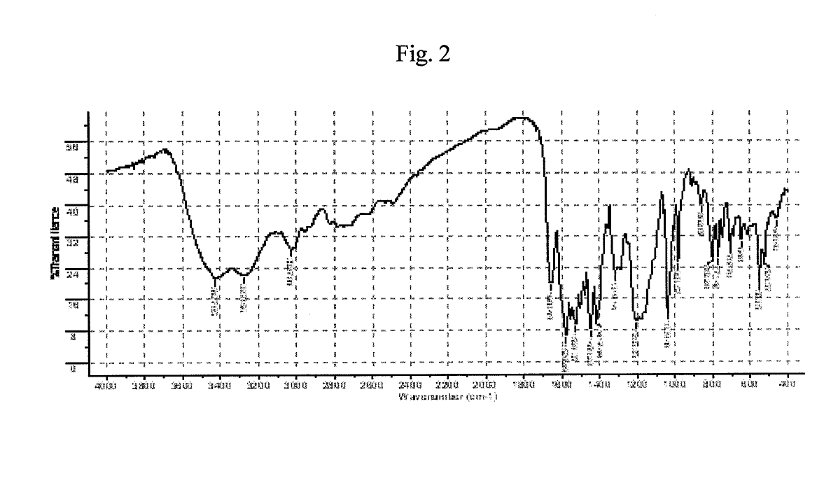

FIG. 3 depicts the TGA curve of amorphous imatinib mesylate (example 4).

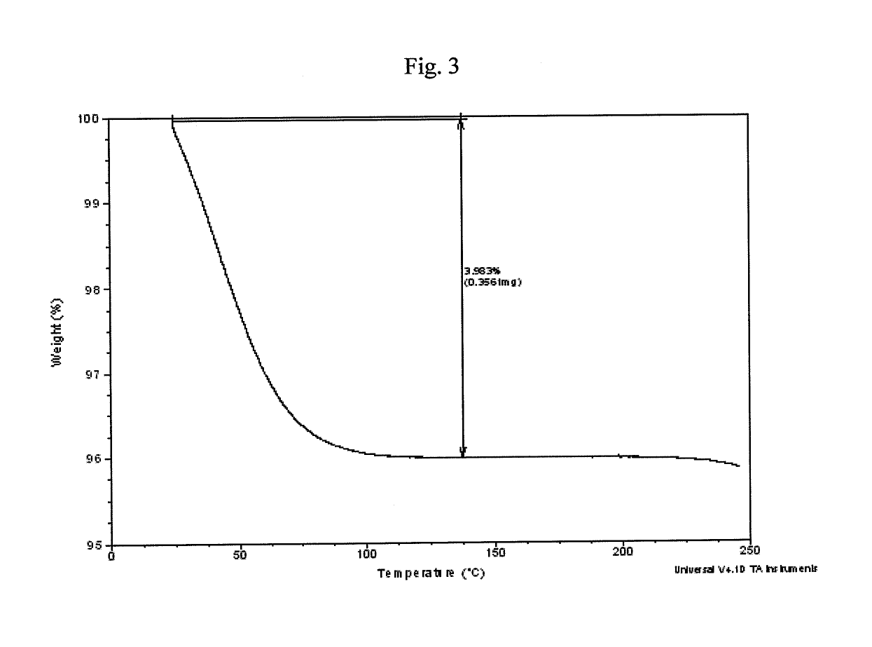

FIG. 4 depicts the X-ray powder diffraction pattern of amorphous imatinib mesylate (example 5).

FIG. 5 depicts the TGA curve of amorphous imatinib mesylate (example 5)

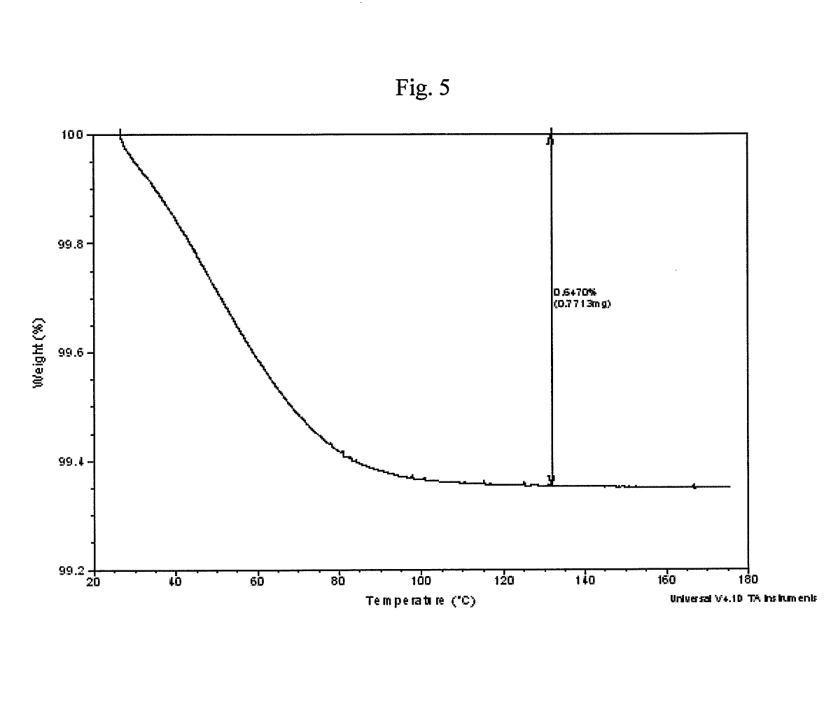

FIG. 6 depicts the X-ray powder diffraction pattern of amorphous imatinib mesylate (example 8).

FIG. 7 depicts the infrared spectrum of amorphous imatinib mesylate (example 8).

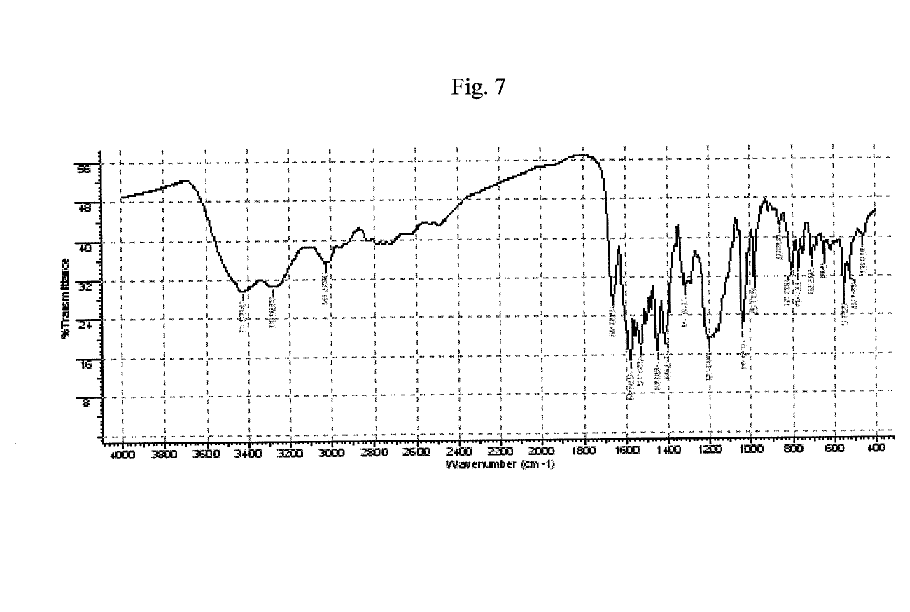

FIG. 8 depicts the TGA curve of amorphous imatinib mesylate (example 8).

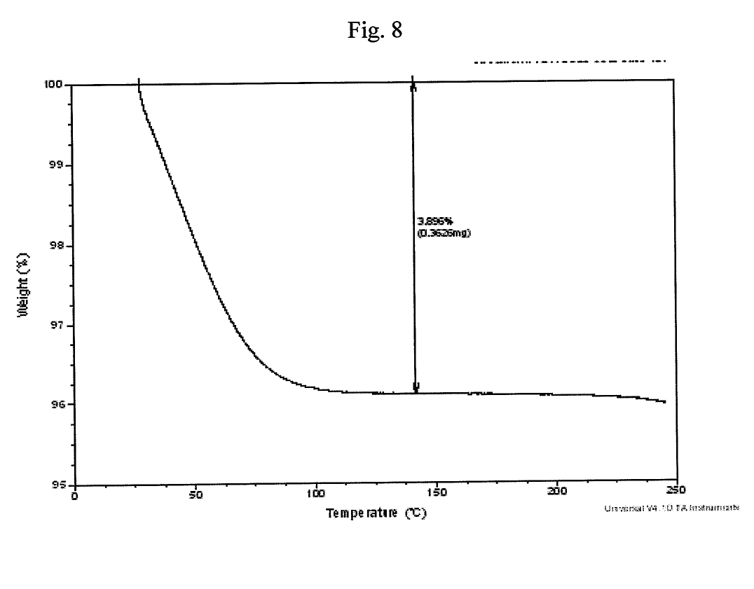

### DETAILED DESCRIPTION OF THE INVENTION

In search for an improved process of preparing stable amorphous imatinib mesylate, applicant has found that stable amorphous imatinib mesylate, having narrow particle distribution, can be prepared by spray drying from an aqueous mixture containing crude imatinib mesylate. The crude imatinib mesylate can be prepared by any method known in the art, including, e.g., the process disclosed in patent application US 2006/0149061 (hereinafter the '061 application). The process disclosed herein is highly applicable for industrial scale-up and is further beneficial since it results in highly pure imatinib mesylate.

In one embodiment, the present invention provides a process for preparing stable amorphous imatinib mesylate, which is highly suitable for pharmaceutical compositions containing the amorphous imatinib mesylate. The process for preparing the amorphous imatinib mesylate includes the steps of:

dissolving imatinib mesylate (obtained, e.g., as described in the '061 application), in water to obtain a solution;

optionally filtering the solution; and

removing the water.

According to another embodiment of the present invention, said removing the solvent can be carried out by a method selected from evaporation, drying, e.g.,

oven drying, spray drying and freeze drying, preferably spray drying or freeze drying.

The crude imatinib mesylate aqueous solution has a concentration of at least 1% by weight, more preferably a concentration of 12% by weight.

The spray dried amorphous imatinib mesylate is a free-flowing solid, having a bulk density of at least 0.1 g/ml, preferably a bulk density of 0.29 g/ml.

The spray dried amorphous imatinib mesylate has a tapped density of at least 0.2 g/ml, preferably 0.39 g/ml (tapped/bulk 1.34).

According to another embodiment of the present invention, 90% of the particles are in the range of less than 50μ, preferably of less than 30μ. According to the data presented in Table 2, the average particle size distribution is D(V, 0.1)=1.5, D(V, 0.5)=8.4, D(V, 0.9)=16.8, hence the span factor is about 1.8:

$$\frac{D [ V , 0.9 ] - D [ V 0 , 1 ]}{D [ V , 0.5 ]} = \frac{16.8 - 1.5}{8.4} = 1.8$$

which demonstrates relatively uniform particle size distribution of a micronized material. Such relatively uniform particle size distribution enables good flowability.

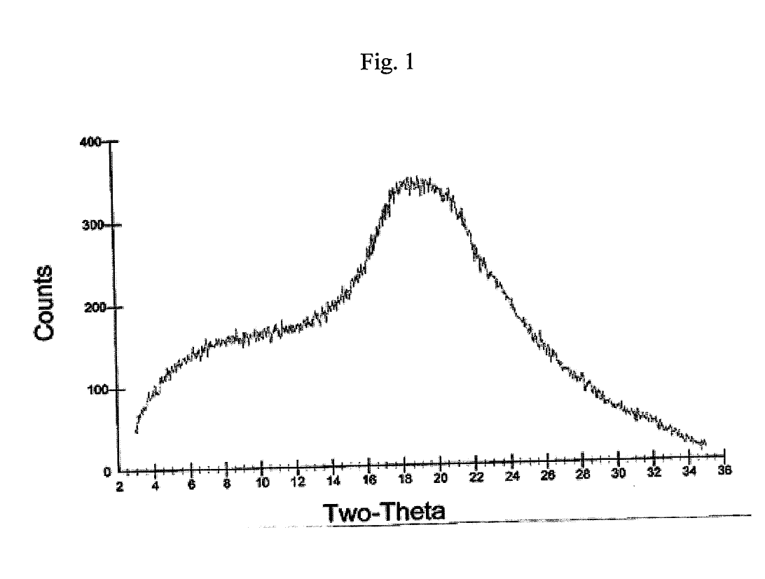

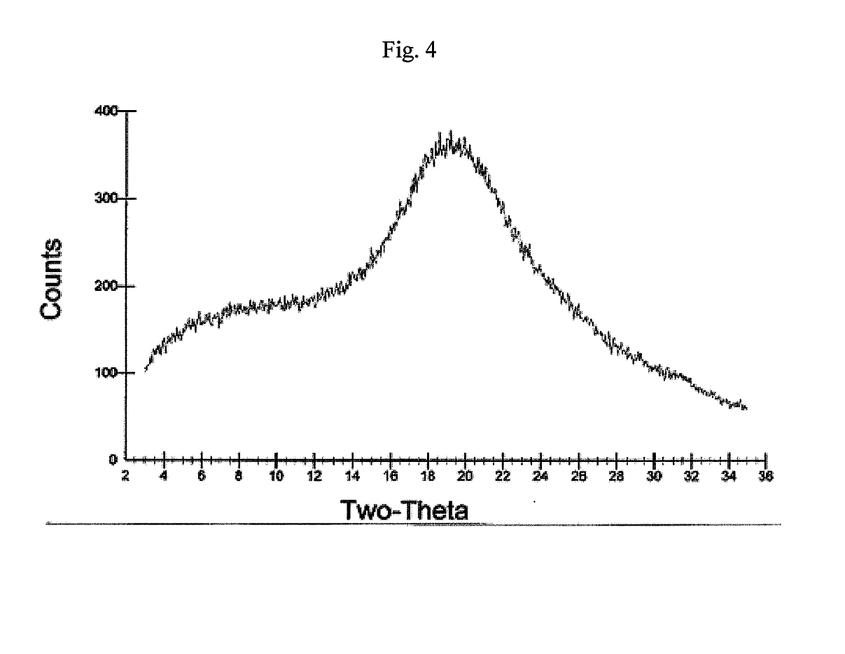

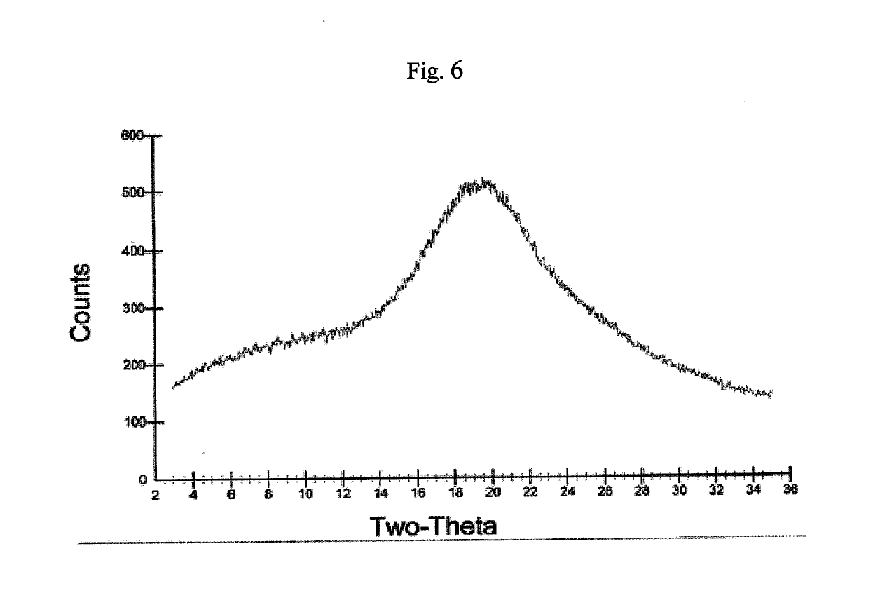

According to another embodiment, the amorphous imatinib mesylate of the present invention produces an X-ray powder diffraction patterns as depicted in FIGS. 1, 4 and 6, which demonstrate its amorphous form.

According to another embodiment, the spray dried amorphous imatinib mesylate of the present invention remains in amorphous form after being compressed under a pressure of about 7.5 tons/cm² for 15 minutes, as checked by X-ray diffractometer.

According to another embodiment, the amorphous imatinib mesylate of the present invention, remains stable and maintains its amorphous form, when stored at 40° C. for a period of at least a month.

According to another embodiment, the amorphous imatinib mesylate of the present invention remains stable and maintains its amorphous form when stored at 25° C. for a period of at least a month.

The term “remains stable”, as defined herein, refers to lack of formation of impurities, while being stored as described herein (see Example 11).

The water content of the spray dried imatinib mesylate of the present invention, which can be determined by the KF method, ranges between 3.2-5.0% (see Table 4). Thus, the spray dried amorphous imatinib mesylate of the present invention is not a hydrate, which contains approximately 3% water.

According to another embodiment of the present invention, highly pure spray dried amorphous imatinib mesylate is obtained by the method provided herein, having a purity of at least 98.5%, preferably having a purity equal to or greater than 99.5%.

In another embodiment, the present invention provides the stable amorphous imatinib mesylate, which is suitable for preparing pharmaceutical compositions. The pharmaceutical compositions according to the present invention include solid oral dosage forms, such as tablets, capsules and the like, which are produced according to regular methods known in the art.

The pharmaceutical compositions of the present invention include pharmaceutically acceptable additives and excipients which are selected from glucose, lactose, manitol, sorbitol, erythritol, maltodextrin, regular or pregelatizined starch, povidone, polyvinylpyrrolidone, carboxymethylcellulose sodium, hydroxyethyl cellulose, hydroxypropyl methyl cellulose, gelatin, guar gum, xanthan gum, citric acid, colloidal silica, colloidal silicone dioxide, sodium silico aluminate, magnesium stearate, polyethylene glycol, propylene glycol, polysorbate 20, 40, 60 or 80, titanium dioxide, and talc.

### EXAMPLES

### Experimental Methods

The following examples further illustrate the invention but, of course, should not be construed as in any way limiting its scope.

General description of the equipment.

X-ray diffraction data were acquired using a PHILIPS X-ray diffractometer model PW1050-70. System description: K_{α1}=1.54178 Å, voltage 40 kV, current 28 mA, diversion slit=1°, receiving slit=0.2 mm, scattering slit=1° with a Graphite monochromator. Measurements of 2θ values typically are accurate to within ±0.2 degrees. Experiment parameters: pattern measured between 2θ=3° and 2θ=30° with 0.05° increments; count time was 0.5 second per increment.

Infrared spectra were run on Nicolet Fourier-transform infrared spectrometer model Avatar 360, with Omnic software version 5.2. All samples were run as KBr disks. The current infrared measurements are accurate to within 4 cm⁻¹.

Spray drying was performed on a mini spray dryer by Buchi model B-290, having heating capacity of 2300 W, evaporative capacity of 1 liter/hour of water, and 1.4 mm spray nozzle.

Particle size was measured on a dynamic light scattering device by Malvern model Mastersizer 2000, using a measuring range of 0.02-2000 μm, accuracy level of 1% at the median, helium neon laser as a red light source, and solid state light source as a blue light source. Particle size distributions are presented as the average particle size of specified percentiles, including the flanking 0.9 quantile of the overall particle size distribution versus volume curve.

Bulk and tapped densities were measured using Varian's Vankel tapped density tester model 50-2300.

The water content measurements were carried out using a Karl Fischer Titrator (Mettler Toledo Model DL-53), according to standard procedures.

Table 1 summarizes the average spray-drying parameters, which afford the highly pure amorphous imatinib mesylate.

TABLE 1
average spray-drying parameters

| No. | Parameter | Value |
|---|---|---|
| 1 | Inlet temperature | 169° C. |
| 2 | Outlet temperature | 88-90° C. |
| 3 | Pump speed capacity | 22-25% |
| 4 | N₂ flow rate | 23 liter/hour |
| 5 | Aspirator rate | 85-100% |
| 6 | Nozzle diameter | 1.4 mm |

### Reference Example 1

This example is a repetition of Example 6 of U.S. Pat. No. 7,300,938. 3.5 g of imatinib mesylate was dissolved in a mixture of 25 ml methanol and 5 ml of water. The solvents were evaporated under vacuum at 50° C. The residue was dried under vacuum using an evaporator during 9 hours to obtain oil, which solidified to a glassy deliquescent material upon long storage. The final water content, according to KF titration, was 2.2% H₂O.

### Reference Example 2

This example is a repetition of Example 8 of U.S. Pat. No. 7,300,938, 3.5 g of imatinib mesylate was dissolved in a mixture of 25 ml methanol and 5 ml of water. The solution was spray dried for 8 hours. The resulting viscous brown liquid was dried for additional 17 hours under vacuum at 50° C. The final water content according to KF titration was 1.7% H₂O. The spray dried material was very electrostatic and tended to agglomerate.

### Example 1

This example demonstrates the preparation of amorphous imatinib mesylate.

A reaction vessel was charged with 9.4 grams of imatinib mesylate, which was prepared as described in the '061 application, and 113 ml of distilled water was added at ambient temperature until complete dissolution was observed. The solution was filtered off and transferred into the spray dryer at the following conditions: Inlet temperature 173° C., N₂ flow 18 liter/hour, pump speed capacity 12%, and aspirator rate 85%. The resulting non-hygroscopic and free-flowing spray dried solid was collected to obtain 5.6 gram of amorphous imatinib mesylate in 59.6% yield, having a purity of 99.6% (according to HPLC). The particle size distribution was D (V, 0.9)=17.2 μm and water content was 4.1% (according to KF).

### Example 2

This example demonstrates the preparation of amorphous imatinib mesylate.

A reaction vessel was charged with 9.0 grams of imatinib mesylate, which was prepared as described in the '061 application, and 30 ml of distilled water was added at ambient temperature until complete dissolution was observed. The solution was filtered off and transferred into the spray dryer at the following conditions: Inlet temperature 200° C., N₂ flow 30 liter/hour, pump speed capacity 5%, and aspirator rate 80%. The bulk density of the obtained amorphous imatinib mesylate was 0.29 g/ml, the tapped density was 0.39 g/ml (tapped/bulk 1.34) and the particle size distribution was D (V, 0.1)=1.1 μm, D (V, 0.5)=5.4 μm and D (V, 0.9)=12.7 μm (Example 2A in Table 2). The particle size distribution of 2 additional samples, obtained according to the procedure described in example 2, are detailed in Table 2 (Examples 2B and 2C).

TABLE 2
Particle size distribution

| Example | D (V, 0.1), μm | D (V, 0.5), μm | D (V, 0.9), μm |
|---|---|---|---|
| 2A | 1.1 | 5.4 | 12.7 |
| 2B | 1.4 | 7.9 | 18.1 |
| 2C | 1.9 | 12.0 | 29.6 |

### Examples 3-10

These examples demonstrate the preparation of amorphous imatinib mesylate.

A reaction vessel was charged with imatinib mesylate, which was prepared as described in the '061 application, and a volume of distilled water, as detailed in Table 3, was added at ambient temperature until complete dissolution was observed. The solution was filtered off and transferred into the spray dryer at the conditions that are detailed in Table 3. The resulting spray dried material was collected to obtain amorphous imatinib mesylate, as detailed in Table 4.

TABLE 3
Spray drying conditions

| | Inlet | Pump speed | N₂ flow rate, | |
|---|---|---|---|---|
| Example | temperature, ° C. | capacity, % | liters/hour | Aspirator, % |
| 3 | 173 | 12 | 18 | 85 |
| 4 | 180 | 12 | 18 | 85 |
| 5 | 172 | 22 | 28 | 90 |
| 6 | 160 | 28-27 | 24 | 100 |
| 7 | 160 | 25-22 | 24 | 100 |
| 8 | 160 | 27-22 | 25 | 100 |
| 9 | 178 | 12 | 18 | 85 |
| 10 | 170 | 23-18 | 27 | 100 |

TABLE 4
Spray drying results

| | Initial | Volume of | | % water in | D (V, 0.9), | Purity by |
|---|---|---|---|---|---|---|
| Example | weight, g | water, ml* | Yield % | the product** | μm | HPLC, % |
| 3 | 9.4 | 113 | 59.6 | 4.1 | 17.2 | 99.6 |
| 4 | 8.6 | 103 | 73.7 | 3.7 | 18.1 | 99.8 |
| 5 | 7.8 | 113 | 37.8 | 4.8 | 16.3 | 99.6 |
| 6 | 17.4 | 209 | 61.0 | 3.8 | 15.2 | 99.7 |
| 7 | 16.1 | 193 | 60.0 | 4.3 | 14.2 | 99.7 |
| 8 | 19.7 | 236 | 68.4 | 3.8 | 14.0 | 99.8 |
| 9 | 4.1 | 49 | 30.0 | 4.9 | 30.1 | 99.7 |
| 10 | 4.1 | 49 | 41.0 | 3.2 | 12.7 | 99.6 |

*The volume of water for dissolving the imatinib mesylate.
**By Karl Fischer titration

### Example 11

This example demonstrate the stability of the amorphous imatinib mesylate

Four samples containing the amorphous imatinib mesylate ex example 3, having purity of 99.6% according to HPLC, with different additives have been prepared by compressing the amorphous imatinib mesylate or its mixture with additives thereof at high pressure, as detailed in Table 5.

The compressing pressure was about 7.5 tons/cm² for 15 seconds.

The four samples, marked as 1-4, were checked by X-ray diffractometer after compressing and the results are detailed in Table 5.

TABLE 5
Tablets containing amorphous imatinib mesylate

| Number | Tablet content | Tablet preparation | Result |
|---|---|---|---|
| 1 | Imatinib mesylate | 500 mg of imatinib mesylate | The material |
| | powder | | remained amorphous |
| 2 | Imatinib mesylate | 250 mg each of imatinib | The material |
| | and lactose | mesylate and lactose | remained amorphous |
| 3 | Imatinib mesylate | 250 mg each of imatinib | The material |
| | and cellulose | mesylate and celullose | remained amorphous |
| 4 | Imatinib mesylate, | 250 mg of imatinib | The material |
| | cellulose and | mesylate and 125 mg each | remained amorphous |
| | lactose | of cellulose and lactose | |

The compressed tablets were packed in a packing comprising internal sealed polyethylene bag, containing the tablets, which was inserted into a middle opaque (e.g., black) polyethylene bag, which was then sealed, wherein the packaging was sealed effectively to prevent penetration of humidity and/or oxygen. The polyethylene bag was packed and sealed in a laminated aluminum bag containing silica.

The samples were packed in the above-mentioned packaging and stored at room temperate and at 40° C. Samples were withdrawn periodically after 5, 8, 13 and 30 days and the purity of the withdrawn material was checked by HPLC.

All the samples tested conserved the amorphous form after being stored at the specified storage period at the specific temperature. Furthermore, the initial chemical purity of the all the four samples marked as 1-4, as detailed in Table 5, did not change over this period, that is, the purity remained 99.6% (according to HPLC) after storage.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein.

1. Stable micronized amorphous imatinib mesylate, having water content in the range of 3.2-5.0%, which is suitable for pharmaceutical compositions containing the amorphous imatinib mesylate.

2. A process for preparing the amorphous imatinib mesylate of claim 1, which includes the steps of:
dissolving imatinib mesylate in water to obtain a solution;
optionally filtering the solution; and
removing the water.

3. The process of claim 2, wherein the said removing is by spray drying or freeze-drying.

4. The process of claim 2, wherein the imatinib mesylate aqueous solution has a concentration of at least 1% by weight.

5. The process of claim 4, wherein the imatinib mesylate aqueous solution has a concentration of about 12% by weight.

6. The process of claim 2, wherein the spray dried amorphous imatinib mesylate is a free-flowing solid, having a bulk density of at least 0.1 g/ml.

7. The process of claim 6, wherein the spray dried amorphous imatinib mesylate is a free-flowing solid, having a bulk density of about 0.29 g/ml.

8. The process of claim 2, wherein the spray dried amorphous imatinib mesylate has a tapped density of at least 0.2 g/ml.

9. The process of claim 8, wherein the spray dried amorphous imatinib mesylate has a tapped density of 0.39 g/ml.

10. The process of claim 2, wherein the particle size distribution of the spray dried amorphous imatinib mesylate is represented by the value of D (V, 0.9), which is less than 50 μm.

11. The process of claim 10, wherein the particle size distribution of the spray dried amorphous imatinib mesylate is represented by the value of D (V, 0.9), which is less than 30 μm.

12. The process of claim 2, wherein the spray dried amorphous imatinib mesylate is obtained having a purity of at least 98.5% (by HPLC).

13. The process of claim 12, wherein the spray dried amorphous imatinib mesylate is obtained having a purity equal to or greater than 99.5% (by HPLC).

14. The process of claim 2, wherein the spray dried amorphous imatinib mesylate remains in amorphous form after being compressed under a pressure of about 7.5 tons/cm² for 15 minutes, as checked by X-ray diffractometer.

15. The process of claim 2, wherein the spray dried amorphous imatinib mesylate remains stable and maintains its amorphous form, when stored at 25° C. as well as at 40° C. for a period of at least a month.

16. A pharmaceutical composition comprising the amorphous imatinib mesylate of claim 1 and pharmaceutically acceptable additives and excipients.

17. The pharmaceutical composition of claim 16, wherein the pharmaceutically acceptable additives and excipients are selected from glucose, lactose, manitol, sorbitol, erythritol, maltodextrin, regular or pregelatizined starch, povidone, polyvinylpyrrolidone, carboxymethylcellulose sodium, hydroxyethyl cellulose, hydroxypropyl methyl cellulose, gelatin, guar gum, xanthan gum, citric acid, colloidal silica, colloidal silicone dioxide, sodium silico aluminate, magnesium stearate, polyethylene glycol, propylene glycol, polysorbate 20, 40, 60 or 80, titanium dioxide, and talc.
